(19) United States
(12) Patent Application Publication
(10) Pub. No.: US 2008/0009070 A1
(43) Pub. Date: Jan. 10, 2008

(54) AMINE DETECTING METHOD

(75) Inventors: Yuh-Jiuan Lin, Taipei (TW); Hong-Ru Guo, Tainan (TW)

Correspondence Address: SUGHRUE MION, PLLC, 2100 PENNSYLVANIA AVENUE, N.W., SUITE 800, WASHINGTON, DC, 20037, US

(73) Assignee: INDUSTRIAL TECHNOLOGY RESEARCH INSTITUTE, Hsinchu (TW)

(21) Appl. No.: 11/682,187

(22) Filed: Mar. 5, 2007

Related U.S. Application Data
(63) Continuation-in-part of application No. 10/262,832, filed on Oct. 3, 2002, now Pat. No. 7,186,799.

(30) Foreign Application Priority Data
Dec. 21, 2001 (TW) 90131930

(51) Int. Cl. G01N 33/68

(52) U.S. Cl. 436/111

(57) ABSTRACT

An amine detecting method. The method includes providing a sensory device coated with an isolated peptide consisting of SEQ ID NO:1, contacting the sensory device with a breath sample, detecting an amine amount present in the sample, and comparing the amine amount with a database to diagnose disorders.

This application is a Continuation-In-Part of pending U.S. patent application Ser. No. 10/262,832, filed Oct. 3, 2002, and entitled “PEPTIDE AND AMINE EXAMINATION METHOD USING THE SAME”.

### SEQUENCE LISTING

| | |
|---|---|
| SEQ ID NO:1 | Asp-Pro-Asp-Gln-Arg-Asp; |
| SEQ ID NO:2 | Gly-Asp-Leu-Glu-Ser-Phe; |
| SEQ ID NO:3 | Glu-Tyr-Asp-Ser-Cys. |

### BACKGROUND OF THE INVENTION

1. Field of the Invention

The invention relates to a bio-detection, and in particular to an amine detecting method.

2. Description of the Related Art

Olfactory proteins are present on the nasal mucosa of mammal olfactory organs. When an olfactory protein reacts with an odorant molecule, a signal is generated, passed through a cascade of biochemical reactions, and delivered to the sensory area in the brain, where the smell is recognized. An olfactory protein has different binding sites for different odorant molecules. A binding site has a unique amino acid sequence that forms a unique three dimensional structure (tertiary structure) to bind the specific odorant molecule. Based on this theory, it is assumed that different amino acid sequences bind to different odorant molecules or odorant compounds with various affinities. Various peptides as the receptor of a sensor can be used to examine the amine content of a sample.

### BRIEF SUMMARY OF THE INVENTION

The invention provides an amine detecting method, in which a sensory device coated with an isolated peptide consisting of SEQ ID NO:1 is provided. The sensory device is contacted with a breath sample. An amine amount present in the sample is detected. The amine amount is compared with a database to diagnose disorders.

Amine compounds have low olfactory threshold, and are especially difficult to detect when present in trace amounts or low concentration. The peptide of the invention features in high binding sensitivity and binding affinity to amine compounds. When the peptide is applied to a sensor for the examination of amine compounds, it provides a sensitive tool for detecting amine, even when the amine is present only in a very low concentration.

A detailed description is given in the following embodiments with reference to the accompanying drawings.

### BRIEF DESCRIPTION OF THE DRAWINGS

The invention can be more fully understood by reading the subsequent detailed description and examples with references made to the accompanying drawing, wherein:

FIG. 1 is a flow chart of the amine examination method using the peptide of the invention.

### DETAILED DESCRIPTION OF THE INVENTION

The following description is of the best-contemplated mode of carrying out the invention. This description is made for the purpose of illustrating the general principles of the invention and should not be taken in a limiting sense. The scope of the invention is best determined by reference to the appended claims.

Many disorders are accompanied by elevated amine levels with detectable presence in the breath. Such disorders include kidney disease, uremia, liver disease, stomach ulcer and others. The disorder therefore can be diagnosed through determining the amine level in the breath. A way to apply the invention in the diagnosis of such disorder is to build a database containing data of normal amine levels in breath and in the subject's breath using the peptide of the invention. When the diagnosis is carried out, the amine content is measured using the peptide of the invention. By comparing the measured amine level to the database, the subject's condition is determined.

The design of the peptide of the invention is based on the analysis of secondary structure of the olfactory protein, and the simulation of the binding site between the tertiary structure of the olfactory protein and the target odorant molecule. The physical and chemical properties of the substance to be examined are also considered. The peptide can be deduced from natural source or be synthetic. Together with a sensor such as a biochip, the peptide is used for detecting amine.

“Amine” or “amine compound” herein means a compound having a NR₃ group. R is an alkyl group or an aryl group. Examples of NR₃ group are trimethylamine, dimethylamine, monomethylamine and ammonia groups.

The peptide of the invention is one of the following:

| | |
|---|---|
| SEQ ID NO:1 | Asp-Pro-Asp-Gln-Arg-Asp, |
| SEQ ID NO:2 | Gly-Asp-Leu-Glu-Ser-Phe, |
| or | |
| SEQ ID NO:3 | Glu-Tyr-Asp-Ser-Cys. |

One or more modifying groups can be added to the C- and/or N-terminals of the peptide if desired. The modifying group can be an amino acid or other functional group. Examples of amino acid include alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine. Examples for functional group include —COOH, —NH2,—CHO, —OH, or —SH group.

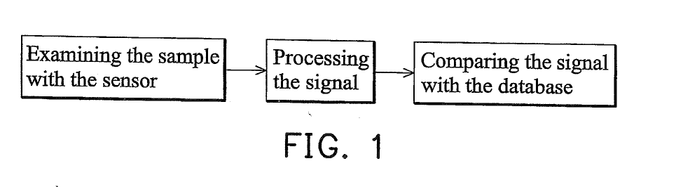

The method of the invention utilizes the previously described peptide to determine the level of amine present in a breath sample. FIG. 1 is a flow chart of the method for detecting amine using the peptide of the invention. The method comprises the application of the sensor coated with the disclosed peptide to examine a breath sample to determine the quantity of the amine presenting therein. The result collected from the sensor is then processed and compared with a database to diagnose disorders. “Amine” herein means a compound having a NR₃ group, wherein R is an alkyl group or an aryl group. The NR₃ group includes trimethylamine, dimethylamine, monomethylamine and ammonia groups.

When the examination is carried out, the sensor is presented with a breath sample. The amine content of the sample is determined based on the binding of the peptide and the amine. The sensory device can be a transducer such as a biochip, piezoelectric quartz crystal, surface acoustic wave, electrochemical, fiber optic, surface plasmon resonance, or metal oxide semiconductor.

Piezoelectric quartz crystal biological sensor is one of the most promising sensors currently in use. In an embodiment, a piezoelectric quartz crystal biological sensor is used. The sensor comprises a piezoelectric quartz crystal and the previously described peptide coated thereon. When the peptide reacts and binds to an amine molecule, the frequency of the piezoelectric quartz crystal changes due to the changed mass. The resulting piezoelectric quartz crystal biological sensor of the invention detects amine with high sensitivity and specificity.

The amine examination method of the invention has a wide variety of applications. For example, the invention can be used to diagnose disorders accompanied by elevated amine levels detectable in the breath. Such disorders include kidney disease, uremia, liver disease, stomach ulcers and others.

Without intending to limit it in any manner, the invention will be further illustrated by the following examples using a piezoelectric quartz crystal as the sensor.

### PREPARATION EXAMPLE 1

Design of the Peptide

Tertiary structure of olfactory protein was used as a template in the computer program “Insight II” to simulate possible binding sites for amine compounds. The selected peptide sequence was then modified according to properties of different amino acids to obtain a peptide sequence that is both specific and sensitive in binding amine. The modified peptide was immobilized on the transducer as a receptor film. The transducer used in the embodiments was a 12 MHz piezoelectric quartz crystal, on which the peptide was coated. The amine examining apparatus was used in determining types and quantity of compounds containing amine groups in a breath sample.

### PREPARATION EXAMPLE 2

Synthesis of the Peptide

The peptide of the invention can be synthesized by conventional peptide synthesis techniques such as solid phase synthesis, liquid peptide synthesis, enzymtic synthesis, or recombinant DNA technology. The peptide used in this example was synthesized by solid phase synthesis using Wang resin as the resin and F-moc as the protecting group in a Peptide Synthesizer (Apply Biosystems, 432A Peptide Synthesizer, USA).

### PREPARATION EXAMPLE 3

Modification of the Peptide and Coating the Peptide on the Piezoelectric Quartz Crystal

A cysteine was attached to the C-or N-terminal of the peptide of the invention. The peptide was then dissolved and diluted in a suitable organic solvent. A 12 MHz piezoelectric quartz crystal with a gold electrode was used in this example. 2-4 μl of the peptide solution was applied to the gold electrode for 4 hours for the peptide to be immobilized on the gold electrode. The cystein at the terminal of the peptide has a —SH group, wherein the sulfur atom forms a very steady covalent bond with the gold molecules of the electrode. The apparatus was ready for use when the detected frequency decrement was between 15000 and 20000 Hz. The procedure of coating the peptide to the sensory device was adjusted according to different properties of the peptide.

### EXAMPLE 1

Diagnosis of Disorders

When the diagnosis was carried out, the breath amine content was measured using the peptide of the invention. By comparing the measured amine level with the database (Table 1), the subject's condition was determined.

The breath ammonia levels of various diseases are listed on Table 1.

TABLE 1

| Disease | Breath ammonia level |
|---|---|
| End-stage renal disease | In the beginning of |
| while they were undergoing | hemodialysis: |
| hemodialysis | 1,500 ppb˜2,000 ppb |
| | At the end of hemodialysis: |
| | 150 ppb˜200 ppb |
| Renal failure | ≧1 ppm |
| Liver cirrhosis | Cirrhotic patients: |
| | 0.745 ppm |
| | Cirrhotic patients with |
| | hyperammonemia: |
| | 0.997 ppm |
| | Cirrhotic patients without |
| | hyperammonemia: |
| | 0.558 ppm |
| Helicobacter pylori | H. pylori-positive subjects: |
| infection | 0.04 ppm ± 0.09 |
| | H. pylori-positive subjects |
| | (increases in breath |
| | ammonia after urea |
| | ingestion): 198˜1,494% |

Various diseases such as kidney disease, uremia, liver disease, and stomach ulcer are determined by corresponding breath ammonia levels recited on Table 1. For H. pylori-positive patients, their breath ammonia levels are lower than H. pylori-negative patients (0.04 ppm+0.09 vs 0.49 ppm ±0.24). However, the H. pylori-positive patients have a significantly greater increase in breath ammonia after urea ingestion (range 198˜1,494% vs 6˜98%). Additionally, for encephalopathy associated with hyperammonemia, expiratory ammonia concentrations in patents with chronic liver disease increase when their blood ammonia concentrations increase to 90 μg/dL above (normal range: 12˜66 μg/dL).

While the invention has been described by way of example and in terms of preferred embodiment, it is to be understood that the invention is not limited thereto. To the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An amine detecting method, comprising:
providing a sensory device coated with an isolated peptide consisting of SEQ ID NO:1;
contacting the sensory device with a breath sample;
detecting an amine amount present in the sample; and
comparing the amine amount with a database to diagnose disorders.

2. The amine detecting method as set forth in claim 1, wherein the sensory device is a piezoelectric quartz crystal, surface acoustic wave, electrochemical sensor, fiber optic, surface plasmon resonance, or metal oxide semiconductor.

3. The amine detecting method as set forth in claim 1, wherein the sensory device is a biochip.

4. The amine detecting method as set forth in claim 1, wherein the isolated peptide is attached at the C- or N-terminus to the sensory device through a cysteine group.

5. The amine detecting method as set forth in claim 1, wherein the amine is trimethylamine, dimethylamine, monomethylamine, or ammonia.

6. The amine detecting method as set forth in claim 1, wherein the disorder is kidney disease, uremia, liver disease, or stomach ulcer.


SEQUENCE LISTING (3 sequences)

SEQ ID NO: 1 - protein, 6 aa, Artificial Sequence
  Peptide for amine detection
DPDQRD

SEQ ID NO: 2 - protein, 6 aa, Artificial Sequence
  Peptide for amine detection
GDLESF

SEQ ID NO: 3 - protein, 5 aa, Artificial Sequence
  Peptide for amine detection
EYDSC